(19) United States
(12) Patent Application Publication
(10) Pub. No.: US 2024/0021975 A1
(43) Pub. Date: Jan. 18, 2024

(54) FLEXIBLE WEARABLE ANTENNA INTENDED FOR WIRELESS COMMUNICATIONS

(71) Applicant: The Research Center for Advanced Materials Science, King Khalid University (KKU), Abha (SA)

(72) Inventors: Abdullah G. Al-Sehemi, Abha (SA); Ahmed A. Al-Ghamdi, Jeddah (SA); Nikolay Todorov Dishovski, Sofia (BG); Nikolay Todorov Atanasov, Sofia (BG); Gabriela Lachezarova Atanasova, Sofia (BG)

(73) Assignee: The Research Center for Advanced Materials Science, King Khalid University (KKU), Abha (SA)

(21) Appl. No.: 17/865,661

(22) Filed: Jul. 15, 2022

(51) Int. Cl. H01Q 1/27; H01Q 1/12

(52) U.S. Cl. CPC H01Q 1/273; H01Q 1/12; A61N 1/37229

(57) ABSTRACT

A flexible wearable antenna intended for wireless communications applicable to medical telemetry. The antenna includes of radiating element fractal-based monopole antenna, made up of electrically mechanically connected fractal-based monopoles, connected to a modified coplanar waveguide transmission line with a conductor element connecting the coplanar waveguide ground planes and of the coplanar waveguide transmission line, wherein the coplanar waveguide center line is connected to the radiating fractal-based monopole and is located between the coplanar waveguide ground planes, wherein the small gaps are located between, the feed point is located between the coplanar waveguide center line and the conductor element, wherein a fractal monopole, coplanar waveguide transmission line and feed point are mounted on the flexible substrate, and the antenna attached to the human body 6 and is connected on both points by a wireless connection to an implanted medical device and/or is connected to a wearable communication device and/or is connected to an external communication device.

### OBJECT OF THE INVENTION

The present invention relates to a flexible wearable antenna intended for wireless communications applicable to medical telemetry.

### PRIOR ART

It is known and made available to the public domain from patent application AU 2021 100 926 A4, published on 29 Apr. 2021, a coplanar waveguide antenna implanted in the human body intended for medical diagnosis of illnesses at an early stage. The antenna has a modified hexagonal structure with a ring placed in the center and radiating edges. The modified hexagonal shaped structure is made of metal raw material and is mounted on a substrate containing epoxy resin.

Patent application U.S. Pat. No. 2,015,048 981 A1, published on 19 Feb. 2015, discloses a wearable dual-band antenna implanted in the human body which consists of: a zeroth-order resonant antenna formed at the bottom of a substrate to receive a signal from a wireless device that is implanted in the human body and a coplanar waveguide antenna formed at the top of the substrate to transmit the signal to a wireless device that is external with respect to the human body.

It is known and made available to the public domain from patent application CN 109 904 602 A, published on 18 Jun. 2019 a flat coplanar waveguide antenna intended for wireless communication with respect to the human body, which is inclusive of two dielectric plates made of epoxy resin.

Patent application JP 2009 106 307 A, published on 21 May 2009, discloses an antenna embedded in the human body, which transmits and receives electromagnetic waves toward and from it, respectively. The antenna is of dielectric type and is made of two copper conductors and dielectric substrates, and the signal transmitted to or received from an external communication device is within the band range from 402 through 405 MHz.

From utility model CN 212 908 060 U, published on 6 Apr. 2021, a wearable antenna based on organic polymeric materials is disclosed and made available to the public domain, which is used for wireless communication with respect to the human body. The antenna mechanism is made of layers of fabric of conductive organic polymers, which replace the metal conductive materials, which makes the antenna lightweight, comfortable, and unnoticeable part of the clothing.

Patent application CN 104 269 615 A, published on 7 Jan. 2015, describes a dual band monopole antenna with the possibility of mounting on the human body, which includes an antenna radiating device, a coplanar waveguide feeding structure and a flexible dielectric substrate. The waveguide feeding structure is equipped with a 50Ω coplanar waveguide center line, and the dielectric substrate constitutes a flexible vinyl polymer. When the antenna is placed on the human body, The structure of the antenna of patent CN 104 269 615 A contains three radiating elements. The antenna substrate of patent CN 104 269 615 A has dimensions of 47.5 mm×28.5 mm and has a frequency band range from 2.40 GHz to 2.51 GHz, the bandwidth is 110 MHz. When placed on a human body model, the efficiency of the antenna is not specified.

### TECHNICAL ESSENCE AND BACKGROUND OF THE INVENTION

The purpose of the invention is to create a flexible antenna that is small in terms of a size, that consists of only one radiating element, which is placed (attached) on the skin surface of a person or animal without causing allergic reactions and without changing the parameters of the antenna, which has a non-directional pattern of directional action that allows communication with respect to one or more communication devices placed on the human body, as well as with a medical device implanted in the human body and/or a remote external communication device.

The object is fulfilled by means of a flexible wearable antenna 1 intended for wireless communications attached to the human body, which consists of a radiating element which constitutes fractal-based monopole 2, composed of electrically mechanically connected fractal-based monopole 2a and 2b, which is connected to a modified coplanar waveguide (CPW) transmission line 3 with a conductor element 3a connecting the coplanar waveguide ground planes 3b and 3c of a coplanar waveguide (CPW) transmission line 3. The coplanar waveguide center line 3d is connected to the radiating element fractal-based monopole antenna 2 and is located between the two coplanar waveguide ground planes 3b and 3c, and the small gaps 3e are located between 3d, 3a 3c. The feed point 4 is located between the coplanar waveguide center line 3d and the conductor element 3a. The fractal-based monopole 2, the coplanar waveguide (CPW) transmission line 3 and the feed point 4 are mounted on the flexible substrate 5. The antenna 1 attached to the human body 6 is connected on both points by means of wireless connection to an implanted medical device 7 and/or is connected to a wearable communication device 9 and/or is connected to an external communication device 8.

The antenna 1 is connected on both points to the implanted medical device 7 by means of a wireless connection 10, is connected on both points to a wearable communication device 9 by means of a wireless communication 11 and is connected on both points to an external communication device 8 by means of a wireless connection 12.

According to an embodiment of the invention, the antenna 1 is connected on both points by means of a wireless connection 10 to the implanted medical device 7.

According to another embodiment of the invention, the antenna 1 is connected on both points by means of wireless connection 12 to the external communication device 8.

The fractal-based monopol 2 has dimensions of 16.5 mm×10.0 mm.

Flexible substrate 5 has the following qualitative and quantitative composition Natural rubber STR 10—100; Zinc oxide—3.0; Stearic acid—2.0; Titanium dioxide—70.0; N-Isopropyl-N′-phenyl-1,4-phenylenediamine—1.0; N-tert-butyl-benzothiasole-sulphenamide—1.5 and Sulfur—2.0.

The conductor radiating element 2 consists of a modified rectangular monopole having a fractal geometric shape formed by means of a Minkowski curve 2a, which is connected to an inverted Minkowski curve 2b, whereby a modified Minkowski curve is created tuned to broadcast in a wide frequency band and covering at least four frequency bands connected to a modified coplanar waveguide (CPW) transmission line 3, intended to match the impedance of the conductor radiating element 2 with the impedance of the coaxial cable. By adjusting the geometric dimensions by changing the length and width of the fractal-based monopole, the length and width of each of the elements of the coplanar line and the small gaps of the coplanar line of the fractal-based monopole 2 and the modified coplanar waveguide transmission line 3, the input impedance of the antenna is matched to the source at the maximum radiation efficiency of the antenna within the desired frequency ranges. The optimal dimensions are selected based on the following criteria, minimum antenna size, maximum transmission efficiency, minimum SAR, maximum bandwidth at the specified input impedance.

Advantages of the antenna according to the invention are:

Radiation efficiency from 4.60% to 4.83% in the frequency range from 2.36 GHz to 2.50 GHz.

The fractal-based monopole 2 has dimensions of 16.5 mm×10.0 mm. The antenna represents only one radiating element; it can be placed (attached) on the skin surface of a person or animal without causing allergic reactions and without changing the parameters of the antenna.

There is a non-directional pattern of directed action, which allows communication with an implanted medical device, inside the human body being suitable for the purposes of a medical telemetry.

Flexible wearable antenna for wireless communications, capable to broadcast in the following frequency bands, namely MBAN (2.36 GHz-2.40 GHz), ISM (2.40 GHz-2.50 GHz), and LTE band 7 (2.50 GHz-2.69 GHz), as well as WLAN frequency band appropriate for indoor use and installations (5.15 GHz-5.35 GHz) Another advantage is that the antenna subject to patent claim has a substrate made of raw materials derived from renewable sources and is environmentally friendly.

### DESCRIPTION OF ENCLOSED DRAWINGS

FIG. 1 Three-dimensional diagram of the flexible wearable antenna

FIG. 2—Embodiment of application of the antenna attached to the human body.

FIG. 3—Second embodiment of application of the antenna attached to the surface of a human arm.

FIG. 4—Third embodiment of application of the antenna attached to the surface of a human arm.

FIG. 5—Embodiment of an antenna mounted on an experimental homogeneous semi-rigid human model.

FIG. 6—Diagram visualizing the transmission coefficient as a function of the frequency of the antenna mounted on an experimental homogeneous semi-rigid human model.

FIG. 7—Second example of the antenna mounted on an experimental homogeneous semi-rigid human model.

FIG. 8—Diagram of the transmission coefficient as a function of the frequency of the antenna mounted on an experimental homogeneous semi-rigid human model.

FIG. 9—Diagram of the reflection coefficient of the antenna mounted on an experimental semi-rigid homogeneous human model.

FIG. 10—Diagram of radiation patterns of the antenna mounted on an experimental semi-rigid homogeneous model.

FIG. 11—Diagram of the efficiency of radiation and gain as a function of frequency (% efficiency) and gain (dBi) as a function of frequency) of the antenna attached to an experimental semi-rigid homogeneous human model.

### EXAMPLES OF EMBODIMENT OF THE INVENTION

The attached examples illustrate preferred embodiments on an illustrative basis with respect to the invention but do not limit to the embodiments described herein.

### Example 1

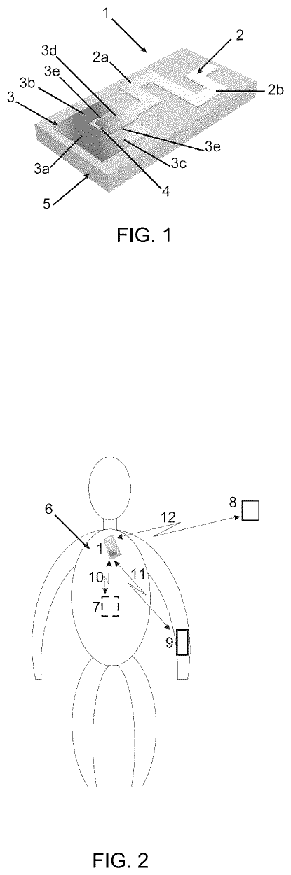

FIG. 1 illustrates the multiband miniature flexible wearable antenna with a polymer substrate 5. The radiating element constitutes a fractal-based monopole 2 is made up of electrically and mechanically connected fractal-based monopoles 2a and 2b.

As a result, small dimensions of the radiating element 16.5 mm×10.0 mm were achieved.

The radiating element 2 is fed by means of a modified coplanar waveguide (CPW) transmission line 3 in order for the conductor elements of the antenna structure to be implemented only on one side of the supporting structure. The modified coplanar waveguide (CPW) transmission line 3 contains a conductor element 3a connecting the coplanar waveguide ground planes 3b and 3c of a coplanar line, and the coplanar waveguide center line 3d connected to a fractal-based monopole 2, which is located between 3b and 3c. The small gaps 3e are located between 3d, 3a 3c. Feed point 4 is located between the coplanar waveguide center line 3d and 3a. Elements 2,3,4 are mounted on a flexible substrate 5 by means of gluing.

According to the invention, a sufficient in terms of extent wideband bandwidth has been achieved to cover three frequency ranges, namely MBAN (2.36 GHz-2.40 GHz), ISM (2.40 GHz-2.50 GHz), and LTE band 7 (2.50 GHz-2.69 GHz), as well as to cover the WLAN frequency band intended for indoor use and installations (5.15 GHz-5.35 GHz) by means of numerical simulations with the finite-difference time-domain method. As a result of the dimensions of the coplanar waveguide, the geometric dimensions of the coplanar waveguide center line 3d and the width of the small gaps 3e, the best combination of small dimensions of the coplanar waveguide 7.5 mm×9 mm is achieved with a characteristic impedance approaching 50Ω (±10%) in respect of the four frequency bands. By means of the feed point 4, an external transmission line connection is provided to the modified coplanar waveguide (CPW) transmission line 3 and the radiating element 2 to transmit a signal from or to the transceiver module of the communication device. The conductor elements of the antenna are mounted on the upper part of a flexible polymer substrate 5 with dimensions of 26 mm×12 mm and thickness of 2 mm. The substrate is specially developed from a mixture of natural rubber with TiO2 filler with electromagnetic parameters, ε′ᵣ=3.16, tan δ=0.0018, σ=0.0008, at 2.565 GHz. By means of the involvement of natural rubber, an easy-to-fabricate flexible wearable antenna with high elasticity and low cost is achieved. The substrate developed does not cause allergic reactions and can be placed on the surface of the skin of a person, and also is composed of products derived from renewable sources and is environmentally friendly. The main function of the antenna substrate relates to its use in terms of a supporting structure, but also its electromagnetic parameters contribute to reducing the size of the antenna. The radiating element and the coplanar waveguide (CPW) can be made of conductive material with a small thickness amounting to of tens or hundreds of microns, such as copper, brass, conductive fabric, conductive ink.

### Example 2

FIG. 2 illustrates an application embodiment of an antenna 1, which is independently mounted on the human body and entails the functions of a coordinator in a wireless body area network (body area network (BAN)) intended for establishing a wireless connection with a medical device implanted in the human body 7 with an external communication device 8 and a communication device 9 placed on a person's arm. The antenna 1 is capable to transmit and receive information from a medical device 7 implanted in the human body by means of a two-way wireless connection 10. The antenna 1 can perform data transfer with a wearable communication device 9 placed on a person's arm by means of a two-way wireless connection 11, as well as to transmit data outside a person's body to a external communication device 8 for real-time analysis of the information via a two-way wireless connection 12. The implanted medical device 7 may constitute implanted cardiac pacemakers or neuromuscular stimulators that help recovery of sensation, mobility and other functions of limbs and organs The disclosed illustrative application can be used for real-time tracking and monitoring of a person's vital physiological status signs.

### Example 3

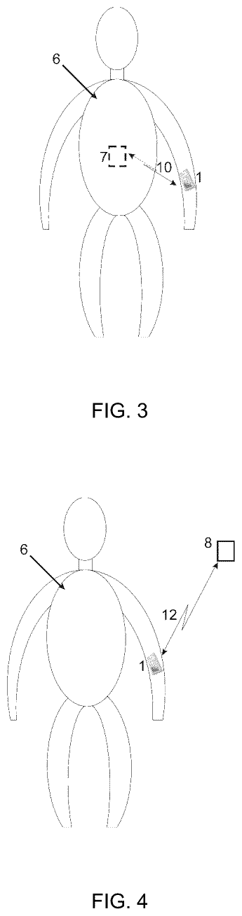

FIG. 3 illustrates an application of an antenna 1 which is independently mounted on a human arm 6 and wireless connection is enabled with a medical communication device 7 implanted in the human body.

According to the invention, the antenna 1 can transmit and receive information from the implanted medical device 7 by means of a two-way wireless connection 10. The disclosed embodiment can be used for real-time monitoring and tracking of vital physiological status signs with respect to a person.

### Example 4

FIG. 4 illustrates embodiment of an application of an antenna 1 that can be independently mounted on an arm of a human body 6 and transmits data outside the human body to an external communication device 8 for real-time information analysis by means of a two-way wireless connection 12. The disclosed embodiment of a possible application can be used for real-time tracking and monitoring of important and vital physiological status parameters and signs with respect to a person.

### Example 5

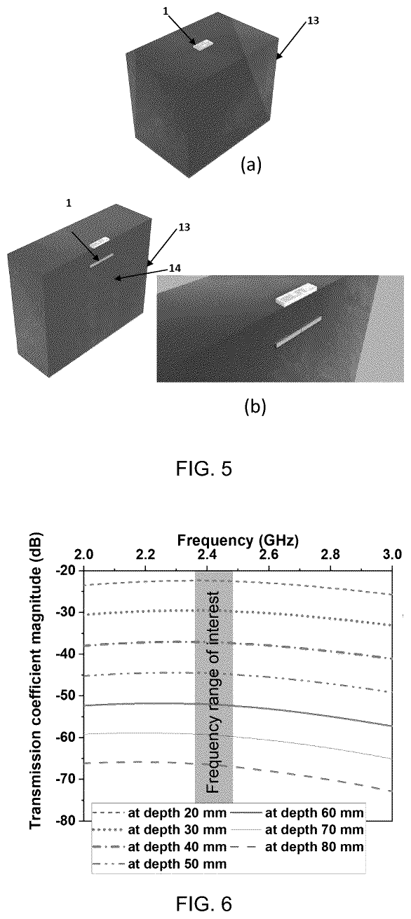

FIG. 5a illustrates an antenna 1 located in the center of a homogeneous semi-solid human model/referred to as phantom/13 having dimensions of 180 mm×150 mm×120 mm and with electromagnetic parameters as follows: real part of dielectric permittivity 43 and conductivity 2.2 S/m, determined at a frequency of 2.565 GHz. An implanted wearable communication device in this embodiment constitutes a dipole antenna 14 located inside a homogeneous semi-rigid human body model 13 under the antenna 1, as shown in FIG. 5b. According to the invention, antenna 1 is in receiving mode and dipole antenna 14 is in transmission mode. The dipole antenna is placed inside the homogeneous semi-solid human body model/referred to as phantom/13 at a distance of 20 mm with respect to the top surface, then moved at a distance of 80 mm from the top surface with a transition step of 10 mm. The results for the transmission coefficient at the different distances between the requested antenna and the implanted one are shown in the diagram represented under FIG. 6. The data in the chart proves that the transmission coefficient is greater than −66 dB in the frequency range 2.36 GHz-2.5 GHz, even when the distance between the requested antenna and the implanted one is 80 mm.

### Example 6

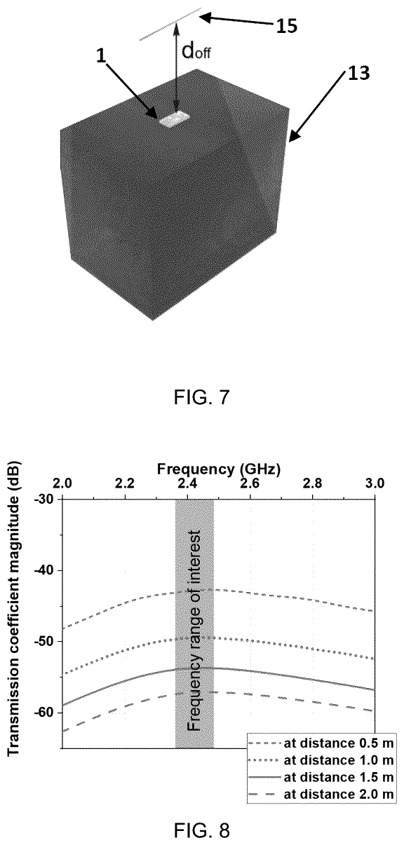

FIG. 7 illustrated an antenna 1 located in the center of a semi-solid homogeneous human model 13 having dimensions of 180 mm×150 mm×120 mm and with electromagnetic parameters, real part of the dielectric permittivity 43 and conductivity 2.2 S/m determined at a frequency 2.565 GHz. An external communication device constitutes a dipole antenna 15, and a wireless connection is enabled with respect to antenna 1, which is in transmission mode, and dipole antenna 15 is in reception mode. The distance between the surface of the semi-solid homogeneous human body model and the dipole antenna is marked in FIG. 7 as d_{off}. The transmission coefficient was measured at four distances 0.5 m, 1.0 m, 1.5 m and 2.0 m. The results for the transmission coefficient at different distances are presented in the diagram which is represented under FIG. 8, the transmission coefficient is greater than −60 dB, even at a distance of 2.0 m, which meets the minimum sensitivity requirements of standard receivers in the frequency ranges of interest 2.36 GHz-2.5 GHz and 5.15 GHz-5.35 GHz.

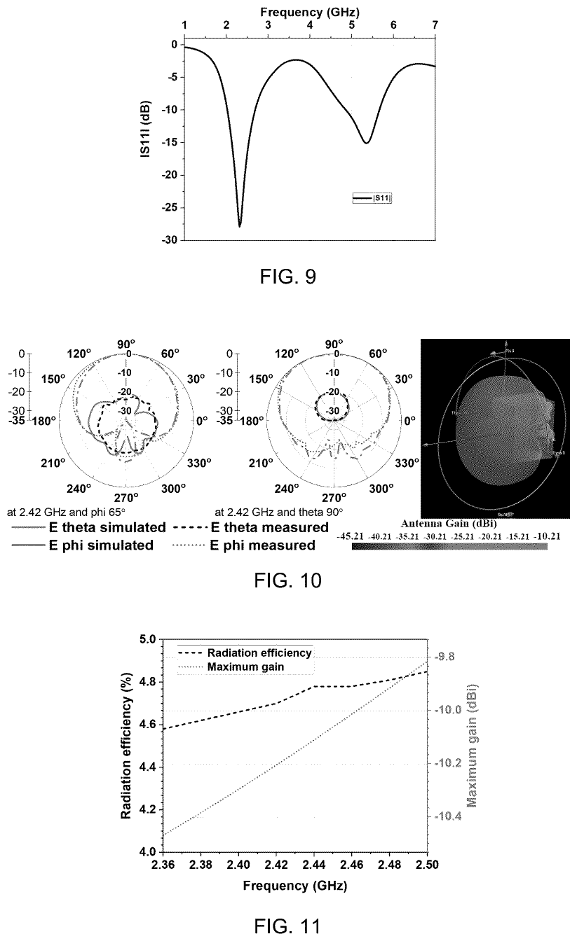

In the diagram represented under FIG. 9 are illustrated the results for the input reflection coefficient of antenna 1 when it is placed on a semi-rigid homogeneous human body model 13. Antenna 1 has an input reflection coefficient lower than −10 dB in the 2.00 GHz frequency bands up to 2.76 GHz and from 4.85 GHz to 5.67 GHz, which to full extent covers the requested frequency ranges, namely MBAN (2.36 GHz-2.40 GHz), ISM (2.40 GHz-2.50 GHz), LTE band 7 (2.50 GHz-2.69 GHz), and WLAN frequency band intended for indoor use and installations (5.15 GHz-5.35 GHz).

The diagrams represented under FIG. 10 illustrate the referred directional pattern at 2.42 GHz, in two planes (xy- and yz-), obtained by the finite-difference time-domain method. Directional action diagrams' patterns at the other frequencies in the frequency ranges of interest showed similar radiation. The antenna according to the invention discloses a non-directional pattern of directional action, which is necessary for the intended application, in wireless communications with communication devices on and off the human body surrounding area.

In the diagram represented under FIG. 11 is illustrated the transmission efficiency in the frequency band from 2.36 GHz through 2.5 GHz and the maximum amplifier gain obtained by the finite-difference time-domain method. The maximum realized gain varies between 10.5 dBi and 9.8 dBi and increases with the advancement of relevant frequency. The negative gain is due to the power absorbed in the homogeneous phantom. The radiation efficiency in the 2.36 to 2.5 GHz frequency range changes slightly from 4.6% to 4.83%, which is suitable for medical telemetry applications. Compared to the flexible antennas presented so far [telecom-02-00019.pdf], wherein the radiation efficiency of the antenna at a frequency of 2.45 GHz is −15.5 dB, in the antenna according to the invention the radiation efficiency at the same frequency is −13.2 dB, which demonstrates 50% higher rate of efficiency. This effect is achieved by means of the antenna according to the invention, which is characterized by smaller dimensions compared to known antennas available to the public domain.

### Example 7

The flexible substrate according to the invention has the following qualitative and quantitative composition Natural rubber STR 10—100; Zinc oxide—3.0; Stearic acid—2.0; Titanium dioxide—70.0; IPPD—N-Isopropyl-N′-phenyl-1,4-phenylenediamine—1.0 and is an anti-aging agent; TBBS—N-tert-butyl-benzothiazole-sulphenamide—1.5 and is a Vulcanization accelerator and Sulfur which constitutes a vulcanizing agent)—2.0.

Flexible substrate 5 has the following electromagnetic parameters ε′ᵣ=3.16, tan δ=0.0018, σ=0,0008 at 2.565 GHz. And is characterized with following dimensions 26 mm×12 mm and thickness amounting to 2 mm.

1. An antenna for wireless communications attached to a person's body comprising:
a coplanar waveguide feeding structure;
a flexible substrate;
a radiating element comprising a fractal-based monopole mounted on the flexible substrate, having a fractal geometric shape based upon a rectangular monopole modified by a Minkowski curve;
a waveguide transmission line mounted on the flexible substrate and connected to the fractal-based monopole;
a feed point mounted on the flexible substrate;
a conductor element connecting two coplanar waveguide ground planes of the waveguide transmission line, wherein a coplanar waveguide center line is connected to the fractal-based monopole and located between the two coplanar waveguide ground planes, the feed point is located between the coplanar waveguide center line and the conductor element, and the antenna is configured to be attached to a human body by means of a wireless connection to at least one of an implanted medical control device, a wearable communication device, and an external communication device.

2. The antenna according to claim 1, wherein the antenna is connected on both points to the wearable communication device.

3. The antenna according to claim 1, wherein the antenna is connected on both points by a wireless connection to the implanted medical device.

4. The antenna according to claim 1, wherein the antenna is connected by means of a two-way wireless connection to the external communication device.

5. The antenna according to claim 1, wherein the fractal-based monopole has dimensions of 16.5 mm×10.0 mm.

6. The antenna, according to claim 1, wherein, the flexible substrate is based per 100 parts by mass on natural rubber and contains the following ingredients Zinc oxide—3.0; Stearic acid—2.0; Titanium dioxide—70.0; N-Isopropyl-N′-phenyl-1,4-phenylenediamine—1.0; N-tert-butyl-benzothiazole-sulphenamide—1.5 and Sulfur—2.0.
